(19) United States
(12) Patent Application Publication
(10) Pub. No.: US 2026/0221225 A1
(43) Pub. Date: Jul. 30, 2026

(54) SOMATIC VARIANT PREDICTION

(71) Applicant: Agency for Science, Technology and Research, Singapore (SG)

(72) Inventors: Kiran KRISHNAMACHARI, Singapore (SG); Anders Martin Jacobsen SKANDERUP, Singapore (SG)

(73) Assignee: Agency for Science, Technology and Research, Singapore (SG)

(21) Appl. No.: 19/146,780

(22) PCT Filed: Jan. 19, 2023
(86) PCT No.: PCT/SG2023/050038

§ 371 Date: Jul. 9, 2025

(51) Int. Cl. G16B 20/20; G06T 7/00; G06T 11/26; G16B 30/10; G16B 40/20; G16B 45/00

(52) U.S. Cl. CPC G16B 20/20; G06T 7/0012; G06T 11/26; G16B 30/10; G16B 40/20; G16B 45/00; G06T 2207/20081; G06T 2207/20084; G06T 2207/30096; G06T 2210/41

(57) ABSTRACT

Method and systems for prediction of somatic mutations based on data of a potentially tumorous sample, by receiving nucleic acid sequencing data of the potentially tumorous sample; transforming the nucleic acid sequencing data into an image-like representation; processing the image-like representation by a trained neural network to predict somatic mutations in the nucleic acid sequencing data.

### TECHNICAL FIELD

This disclosure generally relates to methods and systems for the prediction of somatic variants in nucleic acid sequence data obtained from a biological sample such as a tissue sample or a blood sample etc.

### BACKGROUND

This background description is provided for the purpose of generally presenting the context of the disclosure. Contents of this background section are neither expressly nor impliedly admitted as prior art against the present disclosure.

Identification of somatic mutations from DNA sequencing of tumor samples is valuable for cancer research and the implementation of precision oncology. The process of calling mutations in tumor DNA is convoluted by both biological variation (e.g. tumor heterogeneity) and technical noise (e.g. sequencing errors) in the samples. Existing best-in-class methods for somatic variant calling commonly rely on statistical models of variant allele frequencies in combination with a series of heuristic filters to remove false positives. These methods have been developed through human expert knowledge of DNA sequencing data and tumor biology. It is desirable to provide improved methods for the prediction of somatic variants or provide an alternative to existing methods.

### SUMMARY

Some embodiments relate to a system for prediction of somatic mutations based on data of a potentially tumorous sample, the system comprising:
at least one processor(s);
memory comprising program code executable by the processor(s) to configure the processor(s) to:
receive nucleic acid sequencing data of the potentially tumorous sample;
transform the nucleic acid sequencing data into an image-like representation;
process the image-like representation by a trained first neural network to predict somatic mutations in the nucleic acid sequencing data;
wherein the first neural network is trained using a training dataset comprising:
nucleic acid sequencing data of a plurality of training samples including both tumor samples and non-tumor samples; and
pseudo-labels for the plurality of training samples generated by processing the sequencing data of the plurality of training samples using an ensemble model for somatic variant prediction.

In some embodiments, the ensemble model comprises a plurality of models for prediction of somatic variant, and the pseudo-labels are generated based on a combination of outputs of the plurality of models.

In some embodiments, the trained first neural network is trained to predict at least one of:
single nucleotide variants; or
insertions and/or deletions in the received nucleic acid sequencing data.

In some embodiments, the received nucleic acid data comprises data of a plurality of candidate sites; and
the image-like representation is generated for at least a subset of the candidate sites using information comprising one or more of: raw alignment data, base quality, mapping quality, strand bias and reference base data.

In some embodiments, the trained first neural network processes an image-like representation of nucleic acid sequencing data of a non-tumorous sample associated with the potentially tumorous sample in combination with the image-like representation of the received nucleic acid sequencing data of the potentially tumorous sample to identify the somatic mutations.

In some embodiments, the processor(s) is further configured to generate a heat map in relation to the image-like representation;
wherein the heat map indicates relative importance parts of the image-like representation in prediction of somatic mutation.

The heat map may be generated by performing Guided Backpropagation on the trained first neural network.

In some embodiments, the heat map indicates one or more variant alleles or one or more candidate sites in the nucleic acid data predicted to comprise somatic mutations.

In some embodiments, the processor(s) is further configured to:
train a second neural network for prediction of somatic mutations in Formalin-Fixed Paraffin-Embedded (FFPE) sample;
wherein the second neural network is trained in part based on application of transfer learning using the trained first neural network.

The application of transfer learning may comprise:
performing prediction of somatic mutations in nucleic acid sequencing data obtained from frozen, FFPE samples obtained from tumor samples and corresponding non-tumor samples using the trained first neural network;
treating somatic mutation predictions obtained based on the frozen tumor samples and corresponding non-tumor samples as positive ground truth labels;
treating somatic mutation predictions for the FFPE tumor samples not overlapping with the somatic mutation predictions obtained based on the frozen tumor samples and corresponding non-tumor samples as negative ground truth labels;
training the second neural network using the positive and negative ground truth labels.

Some embodiments relate to a method for prediction of somatic mutations based on data of a potentially tumorous sample, the method comprising:
receiving nucleic acid sequencing data of the potentially tumorous sample;
transforming the nucleic acid sequencing data into an image-like representation;
processing the image-like representation by a trained neural network to predict somatic mutations in the nucleic acid sequencing data;
wherein the first neural network is trained using a training dataset comprising:
nucleic acid sequencing data of a plurality of training samples including both tumor samples and non-tumor samples; and
pseudo-labels for the plurality of training samples generated by processing the sequencing data of the plurality of training samples using an ensemble model for somatic variant prediction.

### BRIEF DESCRIPTION OF THE DRAWINGS

Some embodiments of systems and methods for prediction of somatic variants, in accordance with the present disclosure, will now be described, by way of non-limiting example only, with reference to the accompanying drawings in which:

FIG. 1 illustrates an overview of an approach for the prediction of a somatic variant;

FIG. 2 illustrates graphs of variant calling accuracy on ICGC tumors, FIG. 2 (a),(b) are Precision/recall curves for SNV calling, FIG. 2 (c),(d) are Precision/recall curves for indel calling;

FIG. 3 illustrates graphs of variant calling accuracy across real tumor datasets;

FIG. 4 illustrates impact of VAF and tumor purity levels on somatic variant prediction;

FIG. 5(a) illustrates the inference of model activation with a VarNet encoding (base channel) of an SNV on chromosome 10 in MBL, the candidate position is repeated 5x in both the normal and tumor image, variant alleles are visible at the candidate site in the tumor sample image, FIG. 5(b) illustrates a heatmap visualization showing pixels in the base channel most important to VarNet's deep learning model;

FIG. 6 illustrates model activation at the mutated and non-mutated site, pixel importance scores are computed for individual sites using Guided Backpropagation, FIGS. 6 (a),(b) are illustrate heat map of averaged pixel importance at non-mutated sites for the base and mapping quality channel, respectively, FIGS. 6 (c),(d) illustrate heat map of averaged pixel importance at High-VAF mutated sites for the base and mapping quality channel, respectively;

FIG. 7 illustrates graphs of benchmarking VarNet and NeuSomatic trained on the same cohort;

FIG. 8 illustrates the performance of VarNet in low-alignability regions;

FIG. 9 illustrates Max-F1 scores achieved by methods on synthetic tumors;

FIG. 10 illustrates precision-recall curves for SNV calling on DREAM tumors;

FIG. 11 illustrates precision-recall curves for indel calling on DREAM tumors;

FIG. 12 illustrates benchmarking data on TGEN-COLO829;

FIG. 13 illustrates precision-recall curves for SNV (left) and indel (right) calling on the SEQC2 established benchmark sample derived from a breast cancer cell line and matched lymphoblastoid cell line;

FIG. 14 illustrates performance at variable tumor purity levels;

FIG. 15 illustrates Model activation at mutated and non-mutated sites through heat map visualization of parts of the input;

FIG. 16 illustrates a block diagram of tumor only and normal, tumor tissue based prediction of somatic variants and transfer learning for FFPE samples;

FIG. 17 illustrates a block diagram of tumor only tissue based prediction of somatic variants;

FIG. 18 illustrates a flowchart for the training of a neural network to perform somatic variant prediction;

FIG. 19 illustrates a flowchart for the generation of image like representations of nucleic acid sequence data;

FIG. 20 illustrates a flowchart for transfer learning of a neural network to process data generated from FFPE tumor samples;

FIG. 21 illustrates some components of a system for predicting somatic mutations; and

FIG. 22 illustrates precision-recall curves for SNV calling achieved by a VarNet and prediction model trained using data from only tumor samples.

### DETAILED DESCRIPTION

Embodiments relate to systems and methods for the prediction of somatic variants based on sequencing data obtained from biological samples. The biological samples may comprise tissue samples, blood samples or any other biological material comprising nucleic acids. Identification of somatic mutations in tumor samples is conventionally based on statistical methods in combination with heuristic filters. This disclosure provides VarNet, an end-to-end deep learning approach for the identification of somatic variants from aligned tumor and matched normal DNA reads or from tumor reads independently. VarNet in this disclosure refers to the disclosed systems, methods, and neural networks for somatic variant prediction. VarNet comprises neural networks trained using image-like representations of nucleic acid sequence data. In some embodiments, VarNet was trained using data of 4.6 million somatic variants annotated in 356 tumor whole genomes. VarNet was benchmarked using a range of publicly available datasets, demonstrating performance often exceeding current state-of-the-art methods. Overall, the results demonstrate how a scalable deep-learning approach could augment and potentially supplant human-engineered features and heuristic filters in somatic variant calling.

Machine learning offers a complementary data-centric approach that can exploit the vast amounts of next-generation sequencing data generated today. SMURF (Huang, W. et al. SMURF: Portable and accurate ensemble prediction of somatic mutations. Bioinforma. Oxf. Engl. (2019)) is an ensemble somatic variant caller that uses machine learning and variant features from four distinct variant callers to predict variants. Some embodiments incorporate SMURF to serve as an ensemble model to generate pseudo-labels/pseudo-somatic variant calls that are used for training a neural network. Other alternatives to SMURF may be incorporated to generate the pseudo-labels. Schematics 120 and 1720 illustrate SMURF for generation of pseudo-labels. Neural networks, including deep learning models operating on raw DNA read alignments learn rich representations of reads comprising both their complex interdependencies as well as the sequence context around mutated sites. The embodiments provide neural networks and training methodologies for neural networks for somatic variant calling where variants have to be evaluated in the context of deeper tumor sequencing data, intratumor heterogeneity, and matched normal reads, for example.

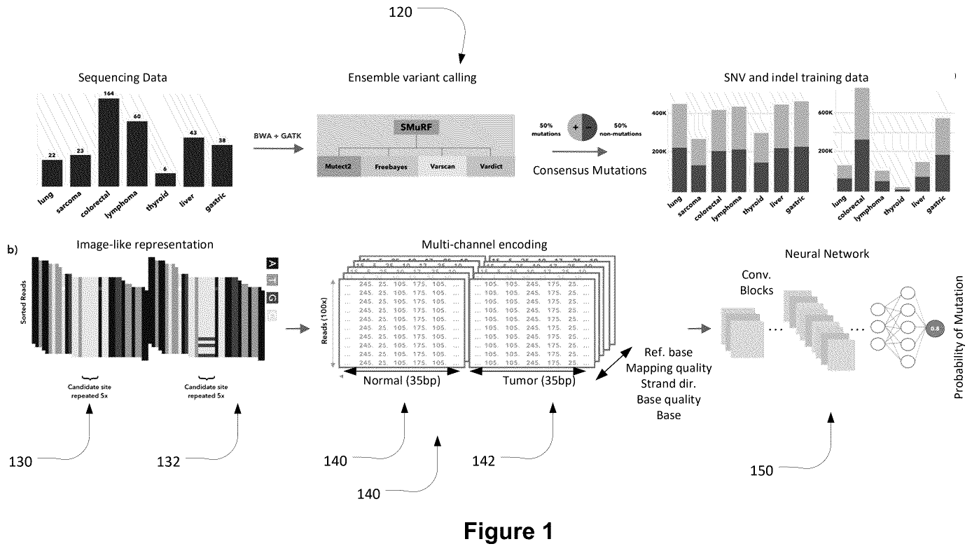

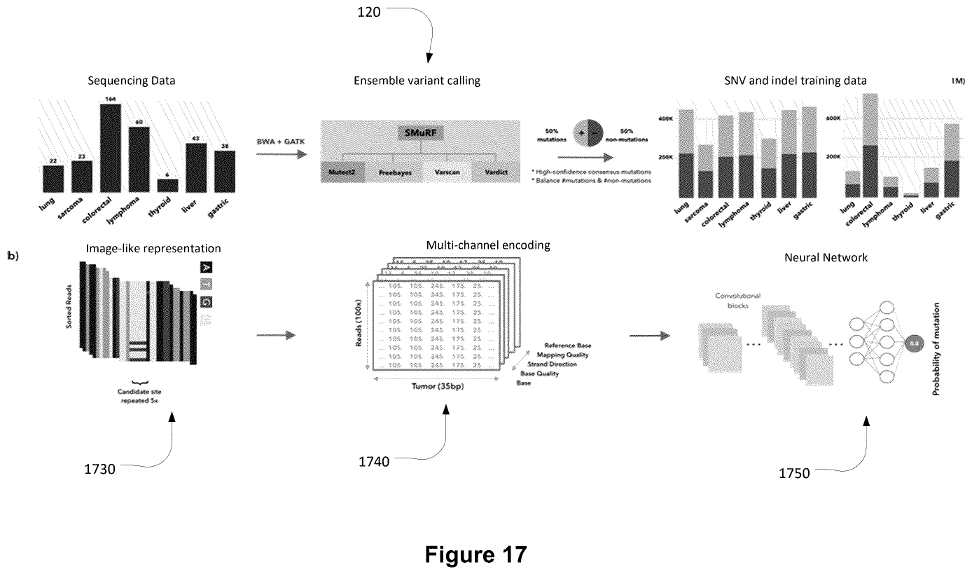

VarNet incorporates deep learning models trained on large amounts of tumor sequencing data to predict somatic single nucleotide variants (SNV) and/or insertions and deletions (indels). VarNet creates image-like representations of aligned reads from known tumor and matched (associated) known normal genomes including their properties such as base quality, mapping quality and strand bias, etc as illustrated in images 130, 132, 140, 142 of FIG. 1. In some embodiments, VarNet creates image-like representations of aligned reads from only known tumor samples as illustrated in images 1730 and 1740 of FIG. 17. The flowchart of FIG. 19 illustrates a method of transforming raw sequence data into image-like representations for further analysis. As supervised deep learning requires access to large labelled datasets, which are typically scarce and expensive to generate in cancer genomics, VarNet uses a weakly supervised learning approach where relatively high-confidence pseudo-labels are generated for tissue samples. The tissue samples may include samples of 7 cancer types and more than 300 cancer whole genomes. Other embodiments may incorporate larger datasets to generate more holistic models. VarNet's performance was evaluated on both real and synthetic tumor benchmark datasets, demonstrating consistent performance often exceeding existing methods.

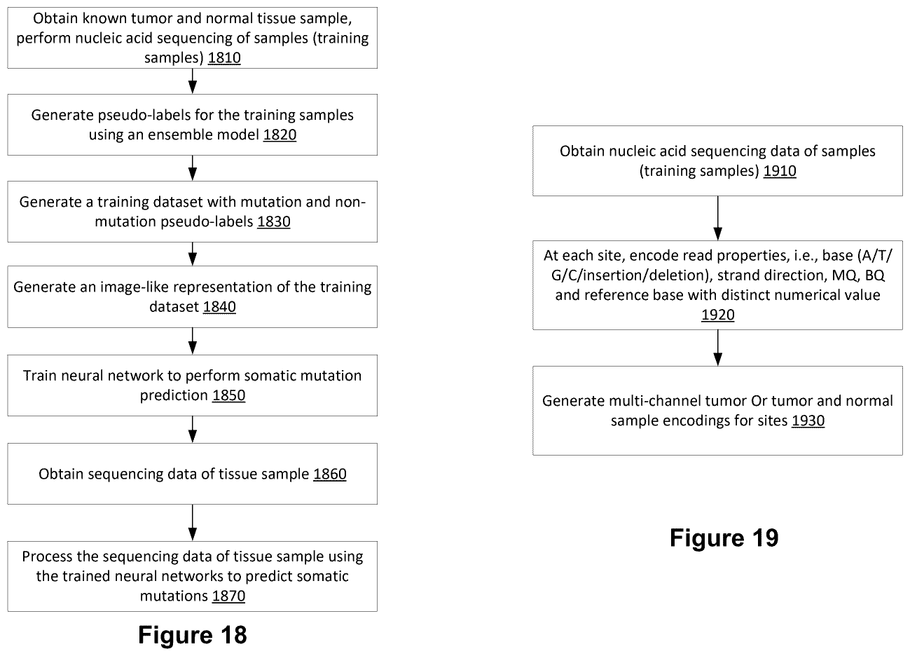

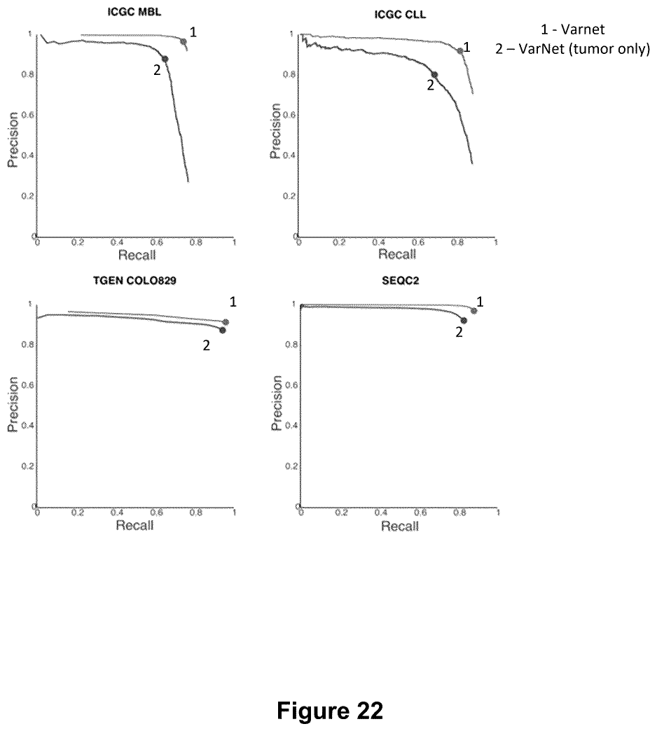

The flowchart of FIG. 18 illustrates a method of training a neural network (for example neural network 150 of FIG. 1 or 1750 of FIG. 17) for the prediction of somatic variants. The method of FIG. 18 is executable by system 2100 of FIG. 21. System 2100 comprises one or more processors 2110 and memory 2120. Memory 2129 comprises program code 2130 that embodiments the logic or instructions of the methods including: methods of somatic variant detection in sequencing data or methods of training a neural network to perform somatic variant detection. VarNet, in some embodiments, was trained on data from over 300 matched (associated) normal and tumor genomes comprising seven cancer types (lung, sarcoma, colorectal, lymphoma, thyroid, liver and gastric cancers). At step 1810, sequencing data of known tumor tissue samples and known non-tumor tissue samples are obtained. The known non-tumor tissue samples are matched to the known tumor samples. The known tumor and/or non-tumor sample comprises genomic data indicative of cell states to guide somatic variant calling. All samples were whole genome sequenced (WGS) at depths of 50-150x. Since ground-truth labels were unavailable, an ensemble method (SMURF) was used to generate mutation calls (SNV and indels) from callsets of four popular mutation callers in the bcbio-nextgen pipeline for somatic cancer variant prediction at step 1820. Training datasets containing relatively comparable numbers of mutated and non-mutated sites were created at step 1830 to train two neural networks (deep learning models, for example). In other embodiments, training data sets were created containing an equal number of mutated and non-mutated sites. The pseudo-labels served as the outcomes or results that the neural networks were trained to detect somatic mutations in sequencing data at step 1850.

One neural network was intended for SNV calling (2.5M sites, for example) and the other neural network was intended for indel calling (2.1M sites, for example) (FIG. 1a). Image-like representations of these sites are generated using the information in raw alignments overlapping these sites including one or more than one of base, base quality, mapping quality, strand bias as well as the reference base, for example were generated at step 1840. FIG. 19 illustrates some steps for generation of the image-like representations. These properties are numerically encoded at each candidate site in distinct input channels along with the surrounding sequence context of neighboring sites so the model can learn relevant mutational signatures of alignment properties. Neural networks (for example deep convolutional networks) were then trained on these image-like representations to predict the probability of mutation at each site. These image-like representations serve as training inputs to the neural networks. Steps 1860 and 1870 relate the application of a trained model to predict somatic mutations in a previously unseen tissue sample. The previously unseen tissue sample may originate from an individual whose tissue may be of interest for detection of somatic mutations. Steps 1860 and 1870 may be performed independently of the rest of the steps of FIG. 18. At step 1860, sequencing data of a tissue sample of the patient may be obtained by the system 2100. At step 1870, the sequencing data is processed by the trained neural networks of the system 2100 to predict the presence of somatic mutations in the sequencing data. The predicted somatic mutations may comprise a prediction relating to SNV in the sequences or indels in the sequence or both. The prediction may serve as an input in developing a genomic profile of an individual or may assist in diagnosis of an aliment in the individual.

### Benchmarking of VarNet's Performance

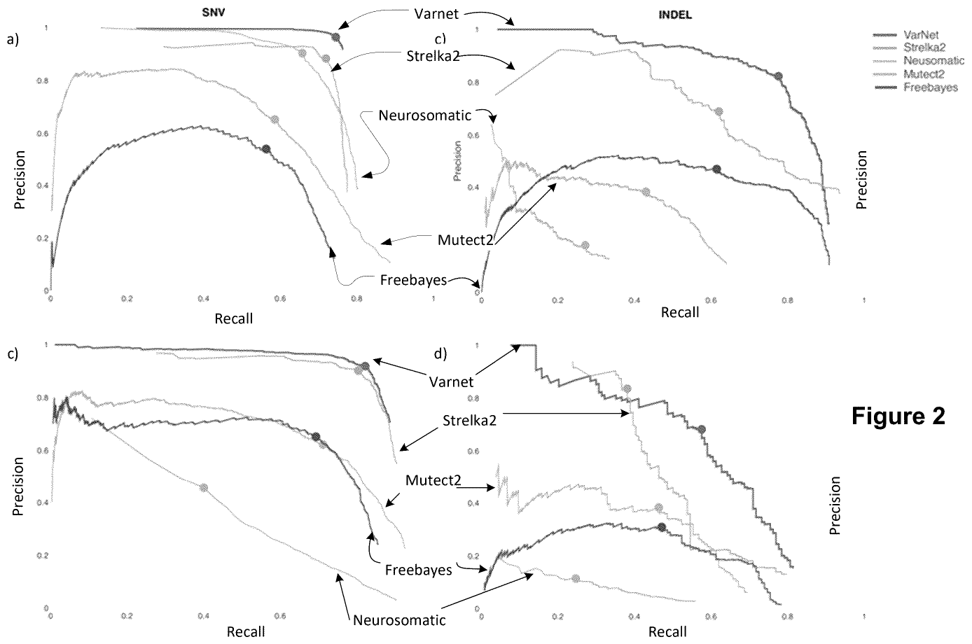

The performance of VarNet was tested on independent and publicly available benchmark datasets comprising both real and in-silico-generated mutations. The International Cancer Genome Consortium (ICGC) Gold Set comprises verified somatic mutations in chronic lymphocytic leukemia (CLL) and medulloblastoma (MBL) tumor-normal pairs that were identified using high-coverage (~300×) whole-genome sequencing (WGS) data from multiple sequencing centres and further curated through manual review. The original high-coverage (~300×) tumor-normal WGS data was downsampled to coverage levels commonly adopted for tumor WGS (~100×). The generalization performance of VarNet was evaluated for both SNV and indel calling on these two samples. Overall, VarNet made calls at higher precision and recall compared to other callers for both SNVs and indels (FIG. 2). On the MBL sample, VarNet outperformed other callers achieving accuracy (F1) scores of 0.84 (SNV) and 0.79 (indel), compared to Strelka2's 0.79 (SNV) and 0.65 (indel), and Mutect2's 0.68 (SNV) and 0.40 (indel). On CLL, VarNet again outperformed other callers achieving F1 scores of 0.87 (SNV) and 0.62 (indel) whereas Strelka2 achieved 0.85 (SNV) and 0.52 (indel). NeuSomatic, which was trained on mutations from a synthetic tumor sample, performed inconsistently on these real tumor samples, achieving F1 scores of 0.43 (CLL) and 0.76 (MBL) for SNV calling, and 0.16 (CLL) and 0.22 (MBL) for indel calling.

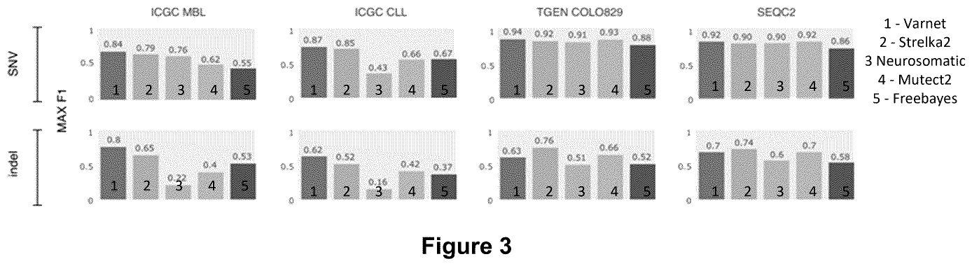

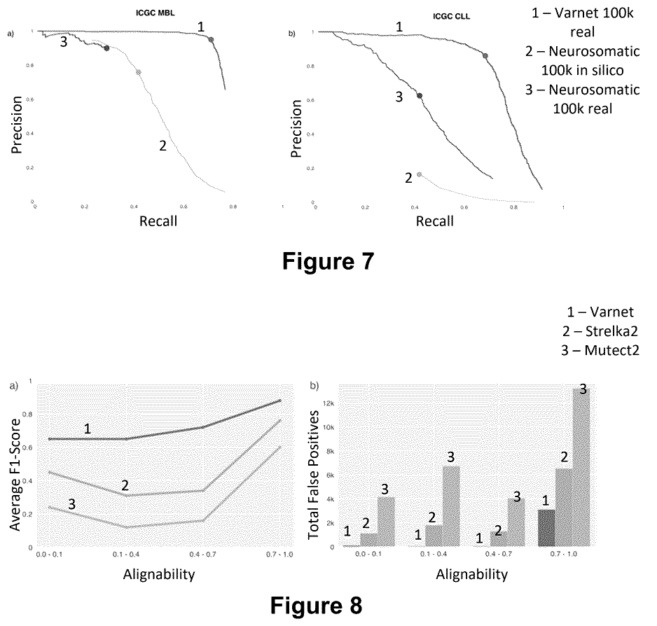

VarNet was also benchmarked on COLO829, a metastatic melanoma cell line with a multi-institutionally defined reference set of somatic mutations, and a SEQC2 established somatic reference callset derived from a breast cancer cell line. These two somatic reference callsets were created by a consensus approach using data from multiple sequencing and variant calling pipelines. The SEQC2 reference callset was partially validated using targeted sequencing (>2,000-fold coverage) to establish high-confidence calls. For SNV calling on COLO829, all callers performed well with VarNet achieving the highest F1-score (0.94) (FIG. 3 and Supplementary FIG. 6a). For indel calling, Strelka2 and Mutect2 achieved higher accuracy (0.76 and 0.66) than VarNet (0.63) (FIG. 3 and Supplementary FIG. 6b). For SNV calling on the SEQC2 reference callset, VarNet achieved the highest F1-score (0.92) along with Mutect2 (0.92) (FIG. 3 and Supplementary FIG. 7). Strelka2 achieved the highest F1-score (0.74) for indel calling followed by VarNet (0.70) and Mutect2 (0.70).

In summary, on SNV calling in real tumor samples, VarNet outperformed all other callers in our analysis (avg. max F1-score=0.89), ahead of current methods such as Strelka2 (0.85) and Mutect2 (0.74). On indel calling, VarNet also often outperformed existing callers with an average F1-score of 0.69, followed by Strelka2 (0.64) and Mutect2 (0.49). Overall, VarNet showed accurate and consistent performance when evaluated on real tumor benchmarks.

### Performance on Low Variant Allele Fraction Mutations

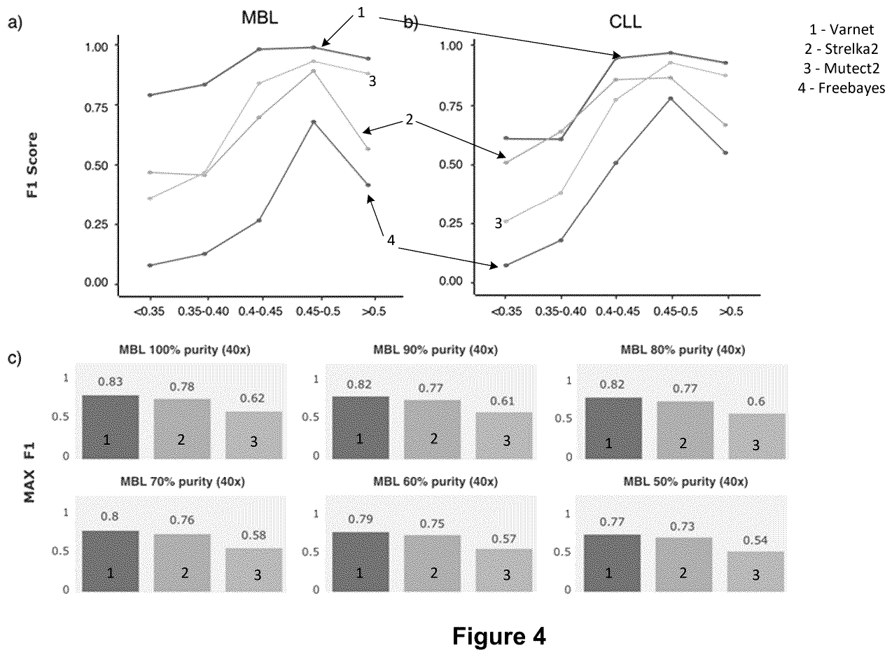

The impact of variant allele frequency (VAF) levels on VarNet's accuracy was evaluated. The MBL tumor sample consists of a tetraploid background combined with altered ploidy at five chromosomes while the CLL tumor sample comprises large copy number variants. This genomic background generated somatic mutations at distinct VAF levels, enabling us to evaluate the performance of VarNet at different VAF ranges (FIG. 4a). For mutations with VAF less than 0.3, VarNet had higher accuracy (average F1 score across CLL and MBL of 0.70) compared to Strelka2 (0.49), Mutect2 (0.31) and Freebayes (0.08). The performance of all methods expectedly improved at higher allele fractions. However, curiously, Strelka2 and Freebayes showed noticeably lower F1 scores at allele fractions greater than 0.5 (29% and 35% lower than 0.45-0.5 range, respectively), potentially mistaking some somatic mutations to be germline mutations at higher VAFs. Overall, VarNet demonstrated high accuracy across both low and high VAF levels as compared to other callers.

### Performance at Low Tumor Purity and Read Depth

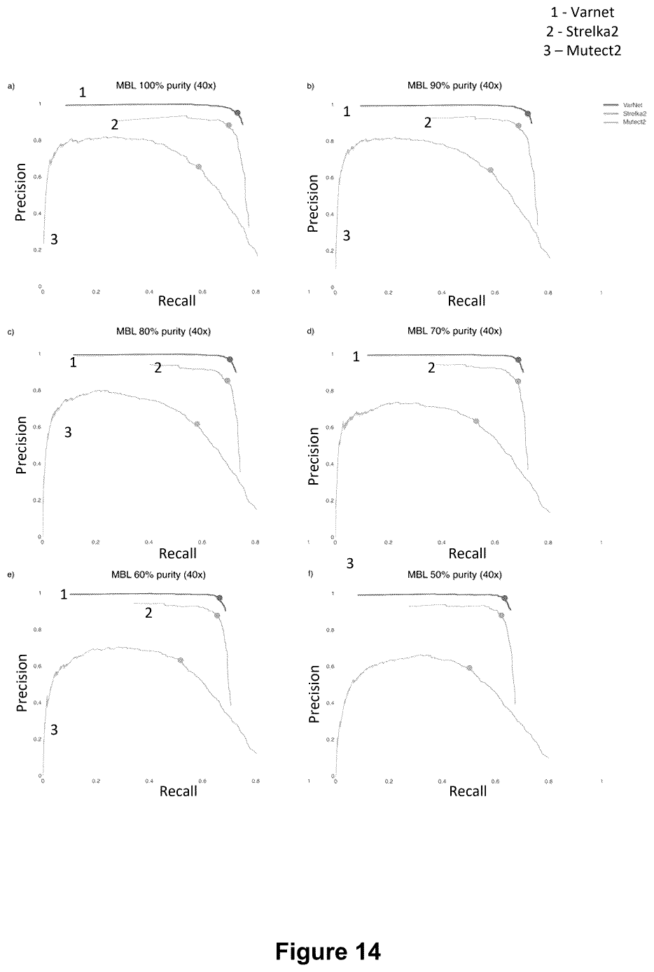

The impact of tumor purity levels (fraction of cancer cells in tumor) as well as low read depths on the performance of VarNet was evaluated. The ICGC MBL sample was estimated to have high tumor purity (>95%) and an average read depth of ~300×. The MBL sample was diluted with reads from the matched normal sample to simulate increasing levels of tumor impurity. In addition, the tumor sample was down-sampled to 40× coverage to simulate low read depth. The lowered read depth alone only had a minor effect on performance for all methods (~1%, FIG. 4c). All methods expectedly demonstrated lower recall with increasing levels of tumor impurity (FIG. 4c). At 70% purity (30% normal dilution), VarNet achieved the highest F1 score (0.80) ahead of Strelka2 (0.76) and Mutect2 (0.58). While VarNet's recall dropped by 7% from the original read depth (~100×) and purity, precision increased from 0.96 to 0.97, suggesting reliable calls even at low read depths and purity levels (FIG. 14). At 50% tumor purity, VarNet still achieved the highest F1 score (0.77) ahead of Strelka2 (0.73) and Mutect2 (0.54). VarNet provided a recall of 0.64 at this lower purity level without any drop in precision (0.97).

### Benchmarking on DREAM Challenge Synthetic Tumor Samples

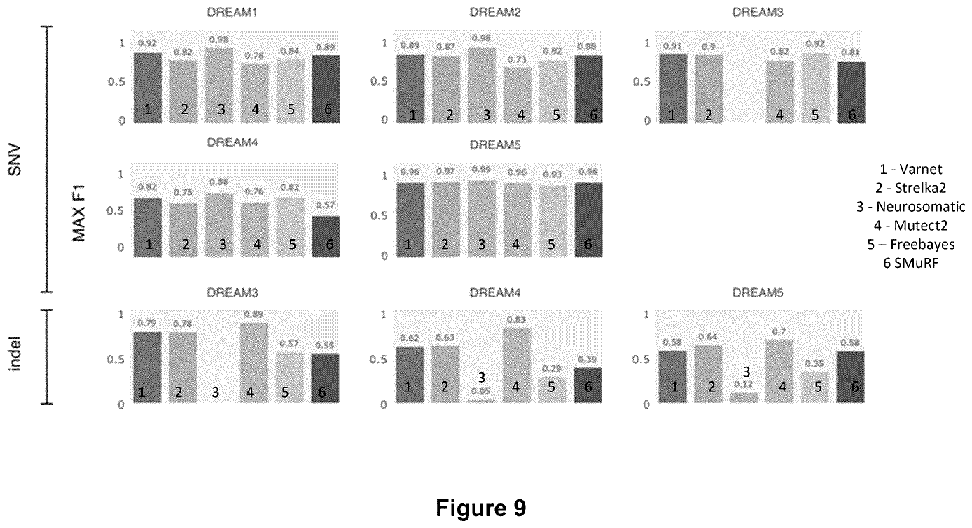

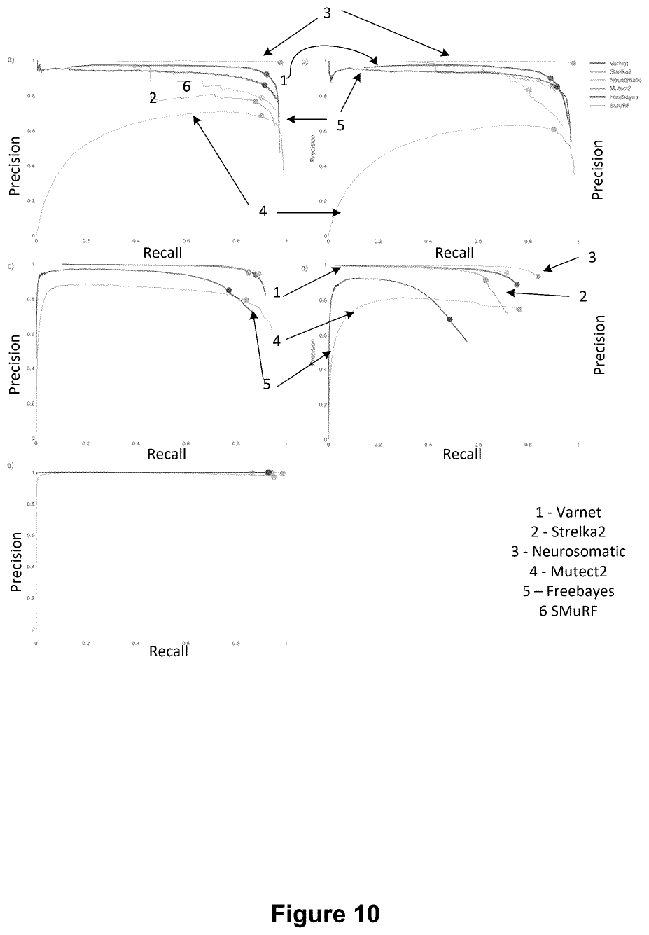

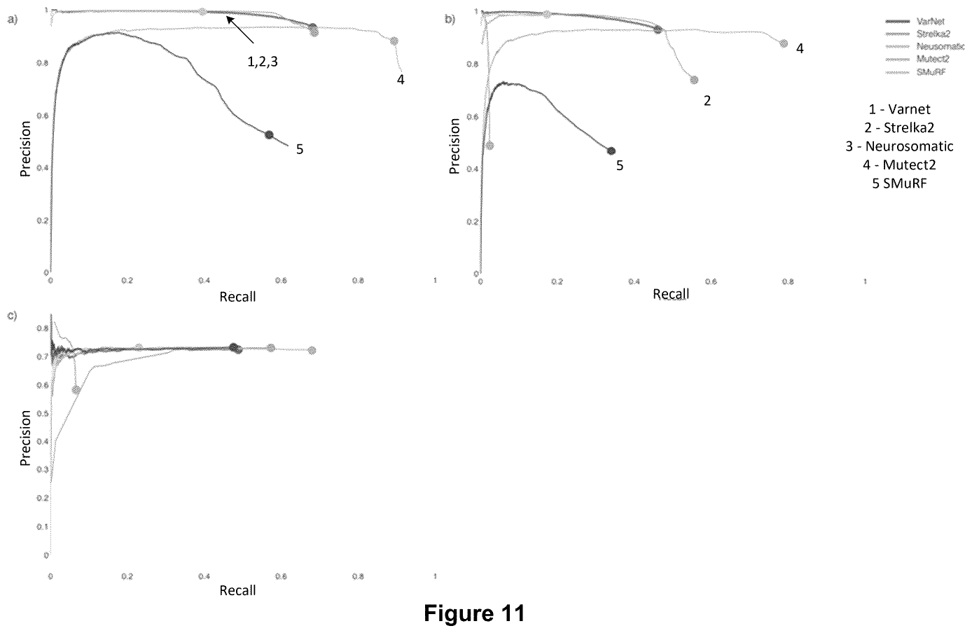

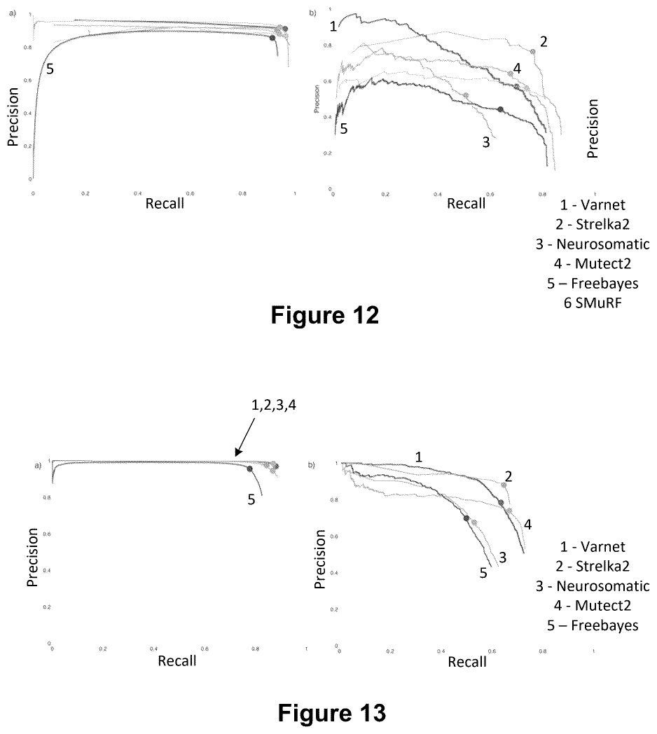

VarNet was also benchmarked on synthetic tumors from the DREAM Somatic Mutation Challenge. This dataset comprises synthetic tumors generated from a cell line sequenced to 80x (split into normal and tumor samples) and where in silico generated SNVs and indels have been added to the tumor sample. For SNV calling, VarNet outperformed most other methods (FIGS. 9, 10). VarNet achieved a top F1 score of 0.90 on average across the synthetic tumors, ahead of Strelka2 (0.86) and Mutect2 (0.81). The only method with better overall performance on the synthetic tumors was NeuSomatic, likely because this method was trained on a subset of the DREAM tumor data. Indeed, while NeuSomatic had the highest SNV calling performance (F1=0.96) across the DREAM synthetic tumors, its performance was not consistent when tested on the ICGC real tumor samples (average F1=0.60, FIG. 2). For indel calling, Varnet achieved an average F1 score of 0.66 across DREAM tumors behind Strelka2 (0.68) and Mutect2 (0.81) (FIG. 11). While Mutect2 achieved best performance for indel calling in the DREAM synthetic tumors, it showed considerably lower performance on the real ICGC tumors (0.41), suggesting a high-variance model. In contrast, VarNet's indel calling performance was consistent across real and synthetic tumors (F1=0.69 versus 0.66). The indel calling performance of Neusomatic was lowest of all methods (average F1=0.09) across the DREAM synthetic tumors. Overall, VarNet performed consistently across real and synthetic tumors, often outperforming existing methods.

### Interpreting Features Exploited by Neural Networks of VarNet

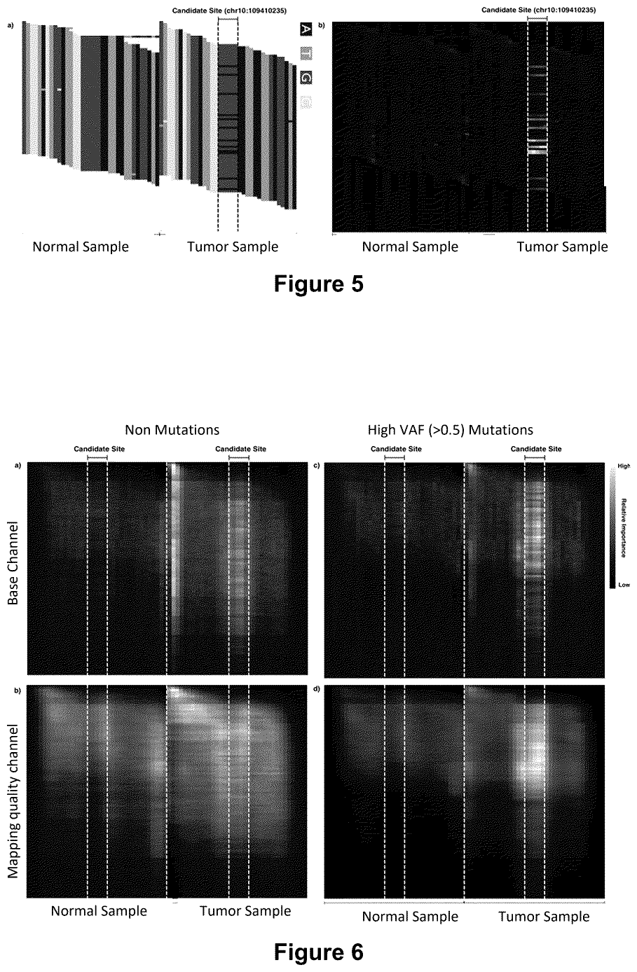

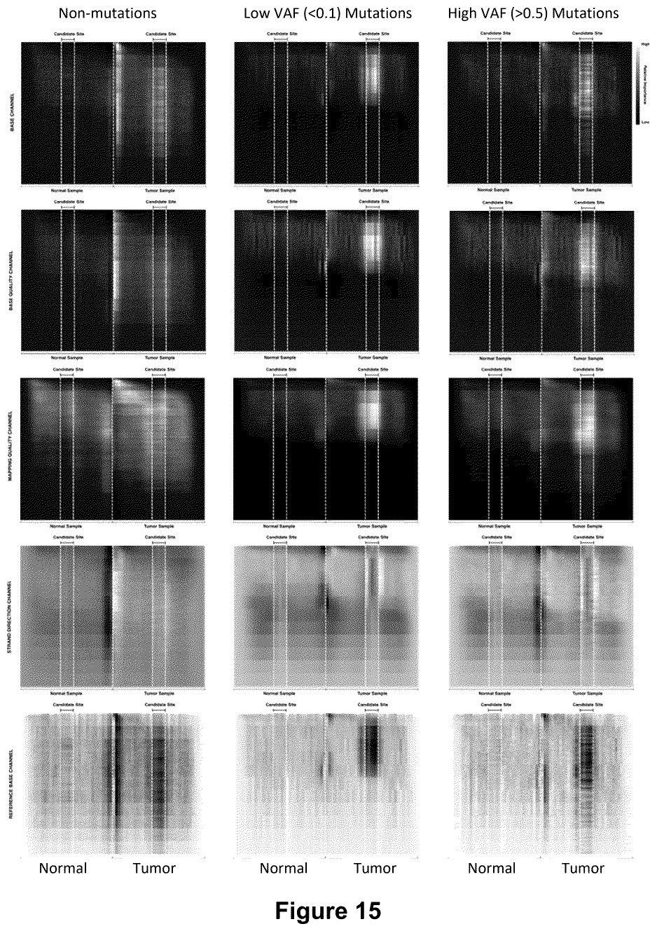

Some embodiments comprise steps to interpret the features learned by VarNet's deep learning model. To aid interpretation, some embodiments generate heat maps of importance assigned by VarNet to individual pixels (regions of image-like representations of sequencing data) in its input using Guided Backpropagation. Guided Backpropagation is a technique that uses model gradients to assign importance scores to regions of image-like representations of the sequencing data (notional pixels). The pixel importance scores were visualized using heat maps to illustrate VarNet's ability to identify variant alleles at an individual mutated site (FIG. 5) as well as an average across many randomly selected sites to interpret commonly used features (FIG. 6). In FIG. 5 and FIG. 6, the relative importance of each site is indicated by a relative brightness of the pixels corresponding to the sites in the image-like representations. Sites of greater importance for prediction are represented with a lighter shade. Sited of a lower importance are represented with a darker shade. Although VarNet's deep learning model was not trained with any specialized knowledge of mutations or genomic data, these visualizations revealed how the model has learned to identify variant alleles at the candidate site in the tumor. In some embodiments, high importance was assigned to pixels containing individual variant alleles at the candidate site across all input channels including base and mapping quality. Activation for the mapping quality feature was evenly distributed across the input image for non-mutated positions (FIG. 6b), but showed higher importance at the mutated site and upstream bases in the presence of mutations (FIG. 6d). Positions upstream of the candidate mutation site are activated across input channels, potentially suggesting use of the immediate sequence and read context by the model. The reference base channel showed higher activation for non-candidate sites suggesting it may not be important for predicting mutations in general and we observed no noticeable differences in pixel activation between low and high VAF mutations (FIG. 15). All input channels indicated highest activation at the tumor candidate site across both mutated and non-mutated inputs (FIG. 15). Overall, these data demonstrate how VarNet uses multiple positions and properties of the encoded alignment images to predict mutations.

Compared to existing callers, VarNet takes a unique approach that does not use human-engineered features to predict mutations. Instead, VarNet incorporates deep learning models trained using rich representations of raw sequence alignments. Genome-wide benchmarking of VarNet was performed on different real tumors and demonstrated best-in-class accuracy and ability to generalize to simulated mutations as well. Moreover, VarNet was able to achieve robust performance in challenging regions of the genome that are not highly alignable. VarNet was also able to outperform the ensemble calling method used to generate pseudo-labels in the training dataset, on independent benchmarks (FIGS. 9, 10, 11). These results suggest that the VarNet is able to successfully learn and generalize when presented with sufficiently large datasets using weak supervision. As additional tumor datasets become available, VarNet in some embodiments leverages more training data through weak supervision or potentially pseudo-labels generated by VarNet itself (self-training) to improve calling performance including indel calling performance.

### Training Data

Training data was generated using WGS tumor data from regional hospitals and research institutes including National University Hospital Singapore, National Cancer Centre (Singapore), Genome Institute of Singapore as well as TCGA (https://www.cancer.gov/tcga). The data repository included 356 matched tumor/normal samples across seven cancer types i.e., lung, thyroid, colorectal, sarcoma, gastric, liver and lymphoma (see FIG. 21). The gastric and liver cancer cohorts were obtained from previously published studies. Samples were sequenced using Illumina HiSeq (Paired-End, medium depth 50-150×) and processed by the bcbio-nextgen pipeline. Reads were aligned to GRCh37 using BWA-MEM followed by marking and removal of duplicate reads. GATK3 with local realignment around indels was used for post-processing.

The pseudo-labels were generated using SMURF, an ensemble somatic mutation caller. Ensembling multiple (noisy) labels is a theoretically justified and practically useful approach for weakly supervised learning. The bcbio-nextgen framework was used to generate somatic variant calls using four callers: MuTect, Freebayes somatic, VarDict and VarScan. Variant and auxiliary features generated by these callers were fed to SMURF, which makes predictions using its random forest classifier.

A total of 2.5 million and 2.1 million training data points were generated for SNV and indel model training, respectively. Fewer indel sites were used for training as indels are generally less common in tumors than point mutations. Both SNV and indel training sets were class-balanced to contain equal numbers of mutated and non-mutated sites (non-mutated sites significantly outnumber mutated sites in any tumor). Calls made by SMURF were chosen as mutated sites while sites that were not called by SMURF, but called by at least one of the four callers, were chosen as non-mutated sites. This improved the quality of non-mutated sites used to train VarNet.

### Cancer-Type Bias and Training Set Size

During training, the trade-offs were performed between down sampling over-represented cancer types (e.g. colorectal cancer) to reduce bias and maintaining the overall size of the training set. While down-sampling over-represented cancer types in the SNV training set improved generalization performance of the SNV model, the indel model benefited from all available training data without balancing cancer types as indels are typically far less frequent than SNVs. Moreover, pseudo-labels for indels were expected to contain more errors than SNVs since variant callers are typically less accurate for indels. Larger training sets can alleviate this label noise when training machine learning models.

### Input Encoding

For each candidate mutation site, aligned reads were encoded in an image-like representation with features including one or more base, mapping quality, base quality and strand bias. FIG. 19 illustrates a flowchart of a series of steps for transforming sequencing data obtained from samples into an image-like representation. The method of FIG. 19 is executable by system 2100 of FIG. 21. Schematics 140, 142 in FIG. 1 and schematic 1740 in FIG. 17 illustrate examples of image-like representations of sequencing data. At step 1910, the nucleic acid sequencing data of samples for transformation into image-like representations is received by the system 2100. This is followed by encoding of the sequence data into an image-like representation.

In the image-like representation, the reference base may be encoded in a separate channel. For example, at step 1920, for each site in the sequencing data, read properties, i.e., base (A/T/G/C/insertion/deletion), strand direction, MQ and BQ are encoded with a distinct numerical value. Deleted bases are also encoded by a unique numerical value different from that of A/T/G/C. Insertions are encoded in-place with adjustments to the reference base channel since inserted bases do not have corresponding loci in the reference. As most short-indels were <10 bp, indels no longer than 35 bp were encoded to fit within the image. In some embodiments, at step 1930, normal and tumor images are encoded adjacently as illustrated in FIG. 1(b). In embodiments where only tumor samples were processed, the tumor sample was encoded independently as illustrated in FIG. 17(b). Image fragments 130, 132 in FIGS. 1 and 1730 in FIG. 17 illustrate examples of fragments of images generated based on a part of the encoding generated at step 1930.

For each site, an input tensor (SNV: (100,70,5), indel: (140,150,5)) encodes the candidate as well as the surrounding sequence context in both tumor and normal so the model is able to learn relevant mutational signatures (FIG. 5a). The candidate site is repeated 5× in the SNV input-encoding to amplify the signal at the candidate mutation site. This was not done for indels as it would affect input-encoding width due to their variable length. The SNV model used up to 100 overlapping alignments while the indel model can use up to 140. If read coverage exceeds this, alignments are randomly sampled. The pysam (https://github.com/pysam-developers/pysam) library, which builds on SAMtools was used to read BAM files and extract alignments.

### Deep-Learning Model and Training

In some embodiments, VarNet incorporated a convolutional neural network (ConvNet) for SNV calling while the Inception V3 architecture was used for indel calling. In some embodiments, the SNV calling model was composed as a convolutional neural network with ten convolutional blocks each containing convolution, ReLu activation and Batch Normalization layers. In some embodiments, two average-pooling layers are used to downsample information between blocks. Convolutional layers were followed by three densely-connected layers (comprising 256, 128 and 64 units) that were followed by a sigmoid output layer that computes the probability of mutation. There are ~3.5 million trainable parameters in the SNV model. For indel calling, a larger model, Inceptionv3, was used. Both models were trained with the Adam optimizer with an initial learning rate of 1e-4 and a mini-batch size of 32. Keras and Tensorflow were used to train models on a Nvidia Titan-X GPU. The above-noted structure, frameworks and hardware are merely exemplary. VarNet may be implemented using alternative structures, frameworks and hardware to perform an equivalent function of somatic variant prediction.

### Genome Pre-Filtering

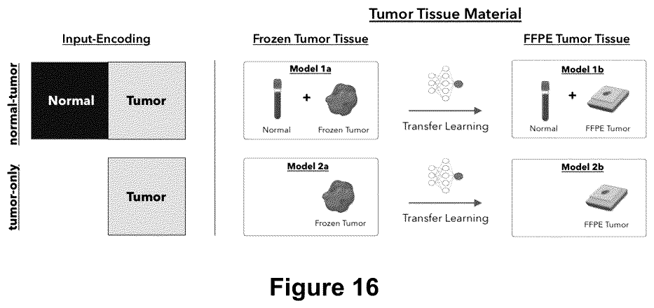

VarNet takes as input binary alignment map (BAM) files of matched tumour-normal pairs. For new samples, as most sites in the sample are unlikely to be mutated (e.g. contain no variant alleles), VarNet first filters positions that have a very low likelihood of being a somatic mutation (FIGS. 16, 17). The goal of this pre-filtering is to reduce computation cost while retaining high sensitivity for mutated sites (FIGS. 18, 19). After filtering, candidate sites are processed using the trained deep-learning models. VarNet also accepts browser extensible data (BED) files of genomic regions to restrict mutation calling and filtering, which is useful for Exome sequencing data.

### Germline Variant Filtering

VarNet performs naive germline variant filtering of its somatic variant callset to remove calls with high likelihood of being germline variants i.e. variant alleles with significant (>10%) representation in the normal sample. This procedure is highly sensitive and filters most germline variants in VarNet's somatic callset while not performing computationally expensive haplotype-based local re-assembly and genotyping. VarNet searches for overlapping germline variants in a 10 bp window around each site in its somatic callset, sufficient to identify germline SNPs and most short-indels in the immediate context. VarNet also filters calls that overlap or are within one base pair of a germline SNP or indel. This filtering procedure is performed as post-processing of the somatic callset, hence, its computational cost is minimal with sensitivity comparable to that of a standalone germline variant caller.

### Performance Metrics

In the reported precision-recall curves for all benchmarks, precision (positive predictive value) refers to the percentage of predicted mutations that are correct, and recall refers to the percentage of true mutations that were correctly identified. We varied scores produced by each caller (VarNet's Score, Strelka2's SomaticEVS, Mutect2's TLOD, Freebayes' ODDS, NeuSomatic's Score, Varscan's SSC) to generate precision-recall curves for each method. The reported F1 scores are a harmonic mean of precision and recall.

### Benchmarking VarNet and NeuSomatic Trained on the Same Cohort

As further tests, VarNet and NeuSomatic models were validated on real tumor samples after training on the same cohort of samples—ICGC MBL and ICGC CLL (FIG. 7). Across both samples, VarNet achieved significantly higher F1-scores (avg: 0.79) than both NeuSomatic-in-silico and NeuSomatic-real. NeuSomatic-in-silico achieved average F1-score 0.39 whereas NeuSomatic-real achieved 0.47. NeuSomatic-real provided less sensitivity and overall F1 performance compared to VarNet-real, which was trained on the same mutations and regions. It is worth noting that training NeuSomatic on real mutations improved over training on in-silico mutations. These results demonstrate the effectiveness of VarNet's model as well as the training methodology using real mutations and weak supervision.

### Performance in Low-Alignability Genomic Regions

While aligners can confidently align reads to most regions of the genome, there are regions that are intrinsically difficult to align due to the presence of repetitive sequences. Alignability is a metric that is used to measure the confidence with which a genomic region can be aligned to. Since it is not possible to confidently align reads in low alignability regions even with high read-coverage or the absence of sequencing errors, callers that make fewer errors and calls in these challenging genomic regions are preferred. For this analysis, experiments with the embodiments used alignability data tracks from the ENCODE consortium to retrieve scores for the human reference genome (https://genome.ucsc.edu/cgi-bin/hgFileUi?db=hg19&g=wgEncodeMapability).

Embodiments used the 75mer track for conservative alignability scores, although we obtained similar results using the 100mer track. All SNV PASS calls made by callers at default filter thresholds (VarNet's SCORE: 0.5, Strelka2's SomaticEVS: 7, Mutect2's TLOD: 6.3) were used to investigate their relative performance in regions of varying complexity. We averaged F1-scores for SNV-calling across three real tumor samples (CLL, MBL and TGEN-COLO829) in regions with different alignability scores. Compared to Strelka2 and Mutect2, VarNet achieved higher F1-scores in low-alignability regions and made most of its FP errors in high-alignability regions with few FPs in low-alignability regions (FIG. 8). VarNet can effectively utilize read mapping-quality scores to make fewer errors in complex regions.

TABLE 1
Filters applied during whole genome pre-filtering to
identify SNV mutation candidates. These candidates
are later processed by the deep learning model

| FEATURE | THRESHOLD |
|---|---|
| MIN READ COVERAGE | 7 |
| MIN MUTANT ALLELE READS IN TUMOR | 2 |
| MIN READ MAPPING QUALITY | 10 |
| MAX MUTANT ALLELE FREQ IN NORMAL | 0.05 |
| MIN MUTANT ALLELE FREQ IN TUMOR | 0.035 |
| MIN BASE QUALITY | 22 |

TABLE 2
Filters applied during whole genome pre-filtering to
identify indel mutation candidates. These candidates
are later processed by the deep learning model.

| FEATURE | THRESHOLD |
|---|---|
| MIN READS WITH INDEL IN TUMOR | 2 |
| MIN READ MAPPING QUALITY FOR | 30 |
| INSERTIONS | |
| MIN READ MAPPING QUALITY FOR | 20 |
| DELETIONS | |
| MIN BASE QUALITY | 22 |
| MIN VARIANT ALLELE FRACTION | 0.03 |

The above noted filters are merely exemplary. Values of the respective filters may be altered to suit different datasets.

TABLE 3
Sensitivity of VarNet's whole genome SNV pre-
filtering on benchmark samples, Pre-filtering identifies
SNV candidates that are processed by the deep learning model

| SAMPLE | TRUE SNVs | SENSITIVITY |
|---|---|---|
| ICGC CLL | 1,319 | 1,292 (0.980) |
| ICGC MBL | 1,263 | 1,166 (0.923) |
| COLO829 | 35,542 | 35,191 (0.990) |
| SEQC2 | 39,447 | 38,350 (0.972) |
| DREAM1 | 3,537 | 3,494 (0.988) |
| DREAM2 | 4,332 | 4,301 (0.993) |
| DREAM3 | 7,903 | 7,720 (0.977) |
| DREAM4 | 16,315 | 15,753 (0.966) |
| DREAM5 | 45,383 | 45,250 (0.997) |

TABLE 4
Sensitivity of VarNet's whole genome indel pre-filtering
on benchmark samples. Pre-filtering identifies indel candidates
that are processed by the deep learning model

| SAMPLE | TRUE indels | SENSITIVITY |
|---|---|---|
| ICGC CLL | 134 | 129 (0.963) |
| ICGC MBL | 347 | 343 (0.988) |
| COLO829 | 446 | 430 (0.964) |
| SEQC2 | 1,625 | 1,575 (0.97) |
| DREAM3 | 7,991 | 7,037 (0.881) |
| DREAM4 | 14,230 | 11,856 (0.833) |
| DREAM5 | 16,428 | 10,839 (0.660) |

TABLE 5
Whole genome run-time requirements for VarNet
under some experiments

| PROCESSING | CPU HRS | MEMORY PER PROCESS |
|---|---|---|
| Genome | 10-12 | 6 GB |
| Filtering | | |
| Prediction | 150 | 10 GB |

TABLE 6
Top F1 scores for SNV calling achieved by VarNet
and VarNet (tumor-only) models

| | ICGC | ICGC | TGEN- | |
|---|---|---|---|---|
| Model/Benchmark | MBL | CLL | COLO829 | SEQC2 |
| VarNet | 0.84 | 0.87 | 0.94 | 0.92 |
| VarNet (tumor-only) | 0.75 | 0.74 | 0.91 | 0.87 |

Embodiments trained using sequencing data from a combination of tumor and non-tumor samples outperformed embodiments trained using sequencing data from only tumor samples as illustrated in FIG. 22 and Table 6.

### Transfer Learning for FFPE Samples

The neural networks of some embodiments may be specifically trained to process nucleic acid sequencing data originating from tissues subjected to Formalin-Fixed Paraffin-Embedding preservation (FFPE) processes. FFPE tissue preservation process introduces artifacts and DNA damage which confounds variant calling and affects the results of somatic variant prediction. Accordingly, the adaptation of a neural network to specifically predict the presence of somatic variants is beneficial for improving the accuracy and performance in predictions. The neural network for processing data originating from FFPE tissue may be trained by the application of transfer learning based on the weights of a neural network trained to predict somatic variants in frozen tissue samples. Frozen tissue samples better preserve DNA in comparison to FFPE tissue. Thus a neural network trained using frozen tissue samples more accurately models the characteristics for prediction of somatic mutations when compared with a neural network trained exclusively using FFPE tissue samples. In addition the FFPE process introduces artifacts and DNA damage which confounds variant calling and affects the results of somatic variant prediction. These disadvantages of FFPE tissue samples are countervailed by the advantages of FFPE tissue samples such as greater durability, ease of storage and multiple clinical applications.

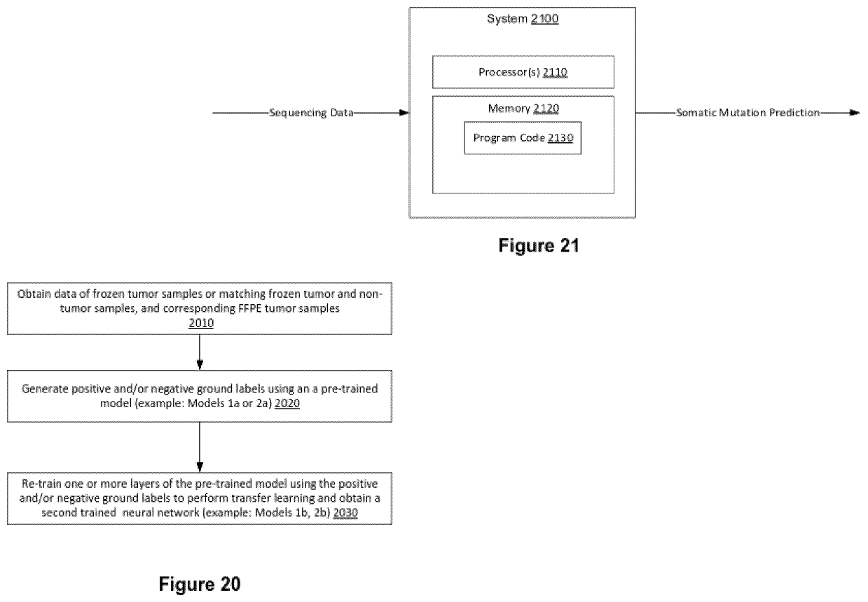

To address the disadvantages of FFPE tissue sample while realizing the benefits of a neural network trained using frozen tissue samples, some embodiments incorporate transfer learning as illustrated in FIGS. 16 and 20. FIG. 20 illustrates steps of a part a part of a method of transfer learning. The steps of FIG. 20 are performed after obtaining a pre-trained VarNet model. FIG. 16 illustrates a schematic diagram that illustrates parts of the steps of the method of FIG. 20. As illustrated in FIG. 16, the steps of FIG. 20 can be performed with reference to either only tumor samples, or both tumor and non-tumor samples. The use of both tumor and non-tumor samples improves the accuracy of the model obtained using transfer learning.

At step 2010, frozen and FFPE tumor/non-tumor samples from a common tumor sample are obtained and sequenced. The origin from common samples allows the analysis of the results of the FFPE preservation process on somatic variant prediction. At step 2020, the pre-trained VarNet model is applied to sequencing data to the samples obtained at 2010. Results obtained by processing the frozen tumor samples are treated as ground-truth (positive) mutation calls. In some embodiments, the positive ground-truth mutation calls may be obtained by processing frozen tumor samples and corresponding non-tumor samples by the VarNet model (example: calls by Model 1a of FIG. 16). As described with reference to FIG. 22 and Table 6, ground-truth (positive) mutation calls obtained based on both tumor and corresponding non-tumor samples have greater accuracy and hence generate more accurate positive ground-truth calls for transfer learning.

Mutation call results obtained by processing the FFPE tumor samples (example: results of Model 1a processing sequencing data from FFPE tumor and non-tumor samples or results of Model 2a processing sequencing data from FFPE tumor only samples) are compared with the results obtained based on the frozen tumor samples (example: results of Model 2a of FIG. 16) or alternatively results obtained using a combination of frozen tumor and corresponding non-tumor samples (example: results of Model 1a of FIG. 16). Non overlapping results (mutation calls) present for the FFPE tumor samples but absent in the positive ground-truth labels are treated as negative mutation calls (negative ground truth labels).

Using the obtained negative and positive ground truth mutation call, at step 2030 the pre-trained model is retrained to perform more accurate somatic variant prediction for FFPE samples. The negative mutation calls represent errors that would have been made by the pre-trained model because of the artifacts introduced in the FFPE sample during preservation. In the schematic of FIG. 16, models 1b and 2b are obtained by performing transfer learning using models 1a and 1b respectively. In alternative embodiments, transfer learning of model 2b may be performed using results obtained from model 1a based on data of both tumor and non-tumor samples; and results obtained from model 2a based data of FFPE tumor samples. The retraining at step 2030 may relate to at least one or more layer of the neural networks of models 1a or 1b.

Similar transfer learning techniques may be applied to train neural networks to perform somatic variant prediction in other tissue types with their own distinctive DNA artifacts introduced because of any processing of the tissue sample. The transfer learning process reduces the need for large training datasets and reduces the training time required to train a neural network for performing somatic variant prediction in a different context.

While this specification generally refers to the training of neural networks for somatic variant prediction, alternatives to neural networks may be incorporated in alternative embodiments to perform the same or similar function. Alternatives to neural networks include support vector machines, random forests, machine learning models based on reinforcement learning techniques etc.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1. A system for prediction of somatic mutations based on data of a potentially tumorous sample, the system comprising:
at least one processor(s);
memory comprising program code executable by the processor(s) to configure the processor(s) to:
receive nucleic acid sequencing data of the potentially tumorous sample;
transform the nucleic acid sequencing data into an image-like representation;
process the image-like representation by a trained first neural network to predict somatic mutations in the nucleic acid sequencing data;
wherein the first neural network is trained using a training dataset comprising:
nucleic acid sequencing data of a plurality of training samples including either tumor only samples or both tumor samples and non-tumor samples; and
pseudo-labels for the plurality of training samples generated by processing the sequencing data of the plurality of training samples using an ensemble model for somatic variant prediction.

2. The system of claim 1, wherein the ensemble model comprises a plurality of models for prediction of somatic variant, and the pseudo-labels are generated based on a combination of outputs of the plurality of models.

3. The system of claim 1, wherein the trained first neural network is trained to predict:
single nucleotide variants; or
insertions and/or deletions in the received nucleic acid sequencing data.

4. The system of claim 1, wherein the received nucleic acid data comprises data of a plurality of candidate sites; and
the image-like representation is generated for at least a subset of the candidate sites using information comprising one or more of: raw alignment data, base quality, mapping quality, strand bias and reference base data.

5. The system of claim 1, wherein the trained first neural network processes an image-like representation of nucleic acid sequencing data of a non-tumorous sample associated with the potentially tumorous sample in combination with the image-like representation of the received nucleic acid sequencing data of the potentially tumorous sample to identify the somatic mutations.

6. The system of claim 1, wherein the processor(s) is further configured to generate a heat map in relation to the image-like representation;
wherein the heat map indicates relative importance parts of the image-like representation in prediction of somatic mutation.

7. The system of claim 6, wherein the heat map is generated by performing Guided Backpropagation on the trained first neural network.

8. The system of claim 6 or claim 7, wherein the heat map indicates one or more variant alleles or one or more candidate sites in the nucleic acid data predicted to comprise somatic mutations.

9. The system of claim 1, wherein the processor(s) is further configured to:
train a second neural network for prediction of somatic mutations in Formalin-Fixed Paraffin-Embedded (FFPE) sample;
wherein the second neural network is trained in part based on application of transfer learning using the trained first neural network.

10. The system of claim 9, wherein application of transfer learning comprises:
performing prediction of somatic mutations in nucleic acid sequencing data obtained from frozen, FFPE samples obtained from tumor samples and corresponding non-tumor samples using the trained first neural network;
treating somatic mutation predictions obtained based on the frozen tumor samples and corresponding non-tumor samples as positive ground truth labels;
treating somatic mutation predictions for the FFPE tumor samples not overlapping with the somatic mutation predictions obtained based on the frozen tumor samples and corresponding non-tumor samples as negative ground truth labels;
training the second neural network using the positive and negative ground truth labels.

11. A method for prediction of somatic mutations based on data of a potentially tumorous sample, the method comprising:
receiving nucleic acid sequencing data of the potentially tumorous sample;
transforming the nucleic acid sequencing data into an image-like representation;
processing the image-like representation by a trained neural network to predict somatic mutations in the nucleic acid sequencing data;
wherein the first neural network is trained using a training dataset comprising:
nucleic acid sequencing data of a plurality of training samples including either tumor only samples or both tumor samples and non-tumor samples; and
pseudo-labels for the plurality of training samples generated by processing the sequencing data of the plurality of training samples using an ensemble model for somatic variant prediction.

12. The method of claim 11, wherein the ensemble model comprises a plurality of models for prediction of somatic variant, and the pseudo-labels are generated based on a combination of outputs of the plurality of models.

13. The method of claim 11, wherein the trained first neural network is trained to predict:
single nucleotide variants; or
predict insertions and/or deletions in the received nucleic acid sequencing data.

14. The method of claim 11, wherein the received nucleic acid data comprises data of a plurality of candidate sites; and
the image-like representation is generated for at least a subset of the candidate sites using information comprising one or more of: raw alignment data, base quality, mapping quality, strand bias and reference base data.

15. The method of claim 11, wherein the trained first neural network processes an image-like representation of nucleic acid sequencing data of a non-tumorous sample corresponding to the potentially tumorous sample in combination with the image-like representation of the received nucleic acid sequencing data of the potentially tumorous sample to identify the somatic mutations.

16. The method of claim 11, wherein the method further comprises generating a heat map in relation to the image-like representation;
wherein the heat map indicates relative importance parts of the image-like representation in prediction of somatic mutation.

17. The method of claim 16, wherein the heat map is generated by performing Guided Backpropagation on the trained first neural network.

18. The method of claim 16, wherein the heat map indicates one or more variant alleles or one or more candidate sites in the nucleic acid data predicted to comprise somatic mutations.

19. The method of claim 11, wherein the method further comprises:
training a second neural network for prediction of somatic mutations in Formalin-Fixed Paraffin-Embedded (FFPE) sample;
wherein the second neural network is trained in part based on application of transfer learning using the trained first neural network.

20. The method of claim 19, wherein application of transfer learning comprises:
performing prediction of somatic mutations in nucleic acid sequencing data obtained from frozen, FFPE samples obtained from tumor samples and corresponding non-tumor samples using the trained first neural network;
treating somatic mutation predictions obtained based on the frozen tumor samples and corresponding non-tumor samples as positive ground truth labels;
treating somatic mutation predictions for the FFPE tumor samples not overlapping with the somatic mutation predictions obtained based on the frozen tumor samples and corresponding non-tumor samples as negative ground truth labels;
training the second neural network using the positive and negative ground truth labels.
